(19) United States
(12) Patent Application Publication
(10) Pub. No.: US 2013/0274602 A1
(43) Pub. Date: Oct. 17, 2013

(54) COLONOSCOPY SYSTEMS AND METHODS

(71) Applicants: Lyndon V. Hernandez, Franklin, WI (US); George K. Lewis, JR., Ithaca, NY (US)

(72) Inventors: Lyndon V. Hernandez, Franklin, WI (US); George K. Lewis, JR., Ithaca, NY (US)

(21) Appl. No.: 13/919,111

(22) Filed: Jun. 17, 2013

Related U.S. Application Data
(63) Continuation-in-part of application No. 13/820,368.
(60) Provisional application No. 61/660,440, filed on Jun. 15, 2012.
(60) Provisional application No. 61/380,065, filed on Sep. 3, 2010.

(51) Int. Cl. A61B 8/12; A61B 8/00; A61N 7/02; A61B 8/08

(52) U.S. Cl. CPC A61B 8/12; A61B 8/085; A61B 8/44; A61N 7/022
USPC 600/438; 600/439; 600/462

(57) ABSTRACT

Systems and methods for performing a colonoscopy or endoscopy using a scope system equipped with both diagnostic and therapeutic ultrasound for more effective colorectal screening, particularly in colons with poor bowel preparation.

### CROSS-REFERENCE TO RELATED APPLICATIONS

This application claims priority to U.S. Provisional Patent Application No. 61/660,440, filed on Jun. 15, 2012 and entitled “Colonoscopy Systems and Methods,” which is incorporated herein by reference.

This application is also a continuation-in-part application of pending U.S. patent application Ser. No. 13/820,368, filed on Mar. 1, 2013, and entitled “Colonoscopy Systems and Methods,” which claims priority to PCT/US2011/050219, filed Sep. 1, 2011, which claims priority to U.S. Provisional Patent Application No. 61/380,065, filed on Sep. 3, 2010, and entitled “A Method of Laxative-Free Colonoscopy,” each of which are incorporated herein by reference.

### STATEMENT REGARDING FEDERALLY FUNDED RESEARCH OR DEVELOPMENT

Not Applicable

### FIELD OF THE INVENTION

The present invention is directed to ultrasound-based systems and methods for performing colonoscopies or endoscopies.

### BACKGROUND OF THE INVENTION

Colorectal cancer is the second leading cancer killer in the United States according to the National Institutes of Health (NIH). In 2007 alone, over 142 thousand Americans were diagnosed and 52 thousand died from colorectal cancer. Globally, over 1.2 million are diagnosed each year, 48 percent of which are not diagnosed early enough and/or treated successfully resulting in morbidity. The success of colonoscopy-based colorectal cancer screening and treatment is strongly dependent on adequate bowel prep, clearing of remaining fecal debris during the procedure, accurate polyp detection, diagnosis and removal, and perhaps most importantly, patient willingness and regular compliance.

A colonoscopy can combine diagnosis and treatment in one session by its ability to remove precancerous polyps, and is expected to remain the most dominant form of screening for several years to come. Colonoscopy uses a lighted motion-controlled tube with intraluminal ports that allows the physician to examine the inside of the rectum and colon in order to inspect for early-stage colorectal cancer prior to symptoms developing. Locating and removing polyps during colonoscopy has been shown prevent colorectal cancer and also reduce deaths from the disease. Though most polyps are benign, some can turn into adenomas and removing them can reduce the risk of developing colorectal cancer by 53 percent.

Colonoscopy screening is the most recognized and efficient methodology for first-line detection of colorectal cancer, and the Centers for Disease Control (CDC) recommends that all men and women aged 50 years or older should be screened regularly for colorectal cancer. However in a 2011 national poll, ⅓rd of adults 60-70 years of age have only been screened once for colorectal cancer, suggesting failure to follow CDC guidelines. The poll also highlighted the fact that individuals found colonoscopy inconvenient, invasive, and unpleasant; bowel preparation was determined to be the most significant obstacle for patients. In addition, the current methods of cleansing can cause abdominal cramping, nausea, vomiting, electrolyte imbalance, and renal failure.

To improve the effectiveness of colonoscopy procedures, water irrigation and evacuation systems have been explored to liquefy remaining fecal matter disguising the colon wall. Current colonoscopies can irrigate the colonic lumen with water during a procedure and evacuate the fluids and unwanted debris using suction applied though the instrument working channel. However, these suction ports are inadequate when the physician is facing a poorly prepped patient with solid stools that cannot be aspirated.

Studies have shown that ultrasound can cause the rapid dissolution of fecal matter in colon phantoms and ex vivo colon models. Studies involving the exposure of fecal matter to ultrasound showed that ultrasound-assisted colonoscopy liquefied stool without insult to the colon wall.

Approximately 14.2 million colonoscopies are done per year in the US. It has been estimated that poor colon cleansing occurs in 20 to 40 percent of colonoscopies, which increases the duration of the colonoscopy by at least 10 percent and the cost of the procedure by up to 22 percent due to repeat visits. The increased cost is due to aborted and inadequate examinations that results from inadequate bowel cleansing. Inadequate bowel cleansing can occur when the patient cannot tolerate the laxative and was not able to finish the prep, or the prep was consumed but is not totally effective (i.e., the laxative failed). Such situations require that patients return at an earlier interval for a repeat colonoscopy. In addition, poor colon cleansing before or during a colonoscopic examination leads to higher rates of missed precancerous polyps.

It would, therefore, be desirable to provide a colonoscopy that avoids the undesirable effects of bowel cleansing prior to a traditional colonoscopy, or can salvage poorly cleansed colons from being aborted, and that can provide both diagnostic and therapeutic ultrasound for more effective colorectal screening, particularly in colons with poor bowel preparation.

### SUMMARY OF THE INVENTION

The subject matter disclosed herein relates generally to systems and methods for performing a colonoscopy, and, more particularly, for performing a colonoscopy using a colonoscopy system equipped with both diagnostic and therapeutic ultrasound for more effective colorectal screening, particularly in colons with poor bowel preparation.

The systems and methods can use focused ultrasound to address the underlying limitation of colonoscopy in practice—lack of easy bowel prep or salvage, and a procedure with rapid biological soft tissue diagnostics. This technology can improve colorectal screening outcomes in tens of millions of procedures world-wide.

According to some embodiments, a medical device is provided. The device includes a flexible tube having an operable portion insertable into a body cavity and a control end. At least one ultrasound transducer is positioned on or in the flexible tube at or near the operable portion of the flexible tube, the ultrasound transducer to generate ultrasound energy. At least one external reverberator is operable in a time reversal acoustic mode. And, an ultrasound generator circuit controls the at least one ultrasound transducer and the at least one external reverberator, the ultrasound generator circuit coupled to the at least one ultrasound transducer and the at least one external reverberator.

According to other embodiments, a colonoscopy system is provided. The system includes a colonoscope having a length, the colonoscope including an ultrasound beacon along the length. A controller is operatively coupled to the colonoscope, the controller including control software and a power amplifier, the control software operable to control ultrasound exposure parameters in a colon treatment area. A reverberator is operatively coupled to the controller, the reverberator operable in a time reversal acoustic mode. And, the ultrasound beacon is operable to focus ultrasound from the reverberator.

According to further embodiments, a method for performing a diagnostic and therapeutic colonoscopy or endoscopy is provided. The method includes, introducing a flexible tube having an operable end insertable into a gastrointestinal tract, the flexible tubing including an ultrasound transducer positioned at or near the operable end of the flexible tube, and at least one water flow channel to deliver water to the gastrointestinal tract, the ultrasound transducer to generate ultrasound energy; applying ultrasound energy to debris located in the gastrointestinal tract; liquefying the debris via the ultrasound energy and water; removing the liquefied debris from the gastrointestinal tract; using time reversal acoustics, focusing ultrasound energy to the ultrasound transducer; and measuring mechanical properties of the gastrointestinal tract tissue for a diagnostic purpose.

The foregoing features and advantages of the invention will appear in the detailed description which follows. In the description, reference is made to the accompanying drawings that illustrate preferred embodiments and wherein like reference numerals denote like elements.

### BRIEF DESCRIPTION OF THE DRAWINGS

FIGS. 1 and 2 show an embodiment of the invention used in an ultrasound liquefaction experiment;

FIGS. 3 and 4 show solid stool in saline pre and post ultrasonic treatment, according to embodiments of the invention;

FIG. 5 is a graph showing a porcine stool liquefaction plot;

FIG. 6 is a view of a colonoscopy system according to an embodiment of the invention;

FIG. 7 is an anatomical view showing a reverberator, according to embodiments of the invention;

FIG. 8 is a view showing an embodiment of a controller for a colonoscopy system, according to embodiments of the invention;

FIG. 9 is a graph showing a porcine focus measurement at 191 kHz and 1 MHz;

FIG. 10 is a perspective view showing an embodiment of the tip of a colonoscope, and showing an embodiment of a polyvinylidene fluoride (PVDF) transducer positioned near the tip;

FIG. 11 is a view showing examples of complex focal volumes obtained from time reversal acoustic (TRA) focusing in a swine colon, according to embodiments of the invention;

FIG. 12 is a graph showing examples of complex focal volumes obtained from TRA focusing, according to embodiments of the invention;

FIG. 13 is a perspective view of an embodiment of a reverberator and showing a plurality of piezoceramics in parallel;

FIG. 14 is an anatomical view showing TRA focusing allowing for controlled ultrasound fields within a colon using a colonoscope according to embodiments of the invention;

FIG. 15 is an anatomical view similar to FIG. 14, showing an embodiment using a diagnostic measurement approach;

FIG. 16 is an anatomical view similar to FIG. 14, showing an embodiment of a temporary sleeve;

FIG. 17 is an schematic view showing an embodiment using a wireless ultrasound capsule; and

FIG. 18 is a flowchart of a method according to embodiments of the invention.

The invention may be embodied in several forms without departing from its spirit or essential characteristics. The scope of the invention is defined in the appended claims, rather than in the specific description preceding them. All embodiments that fall within the meaning and range of equivalency of the claims are therefore intended to be embraced by the claims.

### DETAILED DESCRIPTION

The following discussion is presented to enable a person skilled in the art to make and use embodiments of the invention. Various modifications to the illustrated embodiments will be readily apparent to those skilled in the art, and the generic principles herein can be applied to other embodiments and applications without departing from embodiments of the invention. Thus, embodiments of the invention are not intended to be limited to embodiments shown, but are to be accorded the widest scope consistent with the principles and features disclosed herein. The following detailed description is to be read with reference to the figures. The figures depict selected embodiments and are not intended to limit the scope of embodiments of the invention. Skilled artisans will recognize the examples provided herein have many useful alternatives and fall within the scope of embodiments of the invention.

The following description refers to elements or features being “connected” or “coupled” together. As used herein, unless expressly stated otherwise, “connected” means that one element/feature is directly or indirectly connected to another element/feature, and not necessarily mechanically. Likewise, unless expressly stated otherwise, “coupled” means that one element/feature is directly or indirectly coupled to another element/feature, and not necessarily mechanically. Thus, although embodiments shown in the figures depict example arrangements of colonoscopy devices, additional intervening elements, devices, features or components may be present in an actual embodiment.

In accordance with the practices of persons skilled in the art of computer programming, the present disclosure may be described herein with reference to operations that may be performed by various computing components, modules, or devices. Such operations may be referred to as being computer-executed, computerized, software-implemented, or computer-implemented. It will be appreciated that operations that can be symbolically represented include the manipulation by the various microprocessor devices of electrical signals representing data bits at memory locations in the system memory, as well as other processing of signals. The memory locations where data bits are maintained are physical locations that have particular electrical, magnetic, optical, or organic properties corresponding to the data bits.

The various aspects of the invention will be described in connection with colonoscopy and endoscopy systems and devices capable of both therapeutic and diagnostic procedures. That is because the features and advantages that arise due to embodiments of the invention are well suited to this purpose. Still, it should be appreciated that the various aspects of the invention can be applied to other areas of the body to achieve other objectives as well.

It is to be appreciated that although a focus is on colorectal disease, aspects of the embodiments described herein can have broad relevance to other scope and catheter-based approaches such as esophageal endoscopes, intravenous catheters, and implantable devices where selective ultrasound-focusing may be beneficial. It is also to be appreciated that aspects of the embodiments described herein can also be used for other purposes, such as neuronal drug delivery (by increasing permeability of tissue), thermal ablation and/or emulsification of tissue, location of tumors, ulcers, and other abnormalities, and diagnostic imaging, for example. The technology, approach, and knowledge gained from using complex time reversal acoustic (TRA) focused ultrasound fields may have far reaching medical and research applications.

Embodiments of the invention can optimize the bowel preparation before or during screening, and rapidly locate and evaluate neoplastic lesions accurately. First, ultrasound can be used to rapidly liquefy fecal matter within the colon and rectum; second, TRA focusing of ultrasound can be used to create focal regions of complex shape that can be matched to the entire colon, polyps, or other tissue targets for diagnostic and therapeutic purposes. Thus, embodiments described herein can help overcome the main limitation of colonoscopy in practice—lack of a simple and patient friendly procedure, by using a novel colonoscopy system described herein to remove the need for extensive bowel prep and to detect polyps and/or other abnormalities that are difficult to visualize.

The embodiments described herein could have profound effects on colonoscopy procedures in the prevention and treatment of several colorectal diseases, including some that are devastating to patients. Colon cancer is the second leading cause of cancer-related deaths in Western nations. There are multiple steps to a colorectal examination: fasting and preparation, sedation, getting to the cecum, cleaning off debris, and discovering and then removing polyps. The variation in detection of neoplastic lesions by colonoscopists is partly due to flat polyps, blind spots, and poor preparation. The quality of preparation impacts polyp detection, duration of the procedure, and interval between colonoscopies in screening programs.

Embodiments of the invention can be independent of specific bowel prep compounds or colonoscope optics. Colonoscopies can irrigate the colonic lumen with a water spray of 100 mL/min during a procedure by evacuating liquid debris using suction applied though the instruments intraluminal port. However, this method is grossly inadequate in salvage-cleaning of poorly prepared patients in 20 percent of colonoscopies, and challenging for cleaning an entire colon without bowel prep. Emerging technology using enhanced water spray is also being considered as a salvage tool for poor bowel preparation, however the embodiments described herein can be more efficient and may not interfere with scope maneuverability, such as during retroflexion.

It has been estimated that embodiments of a colonoscopy system described herein can reduce these procedure costs by at least 20 percent. It has been conservatively estimated that within three years, embodiments of the colonoscopy system could be used in at least 100 thousand procedures—this amounts to an estimated $40 million in savings to the health care system per annum. Lack of convenient and adequate bowel prep, combined with a lack of an all-in-one colonoscope for cleansing and screening for neoplastic lesions of the colon, are still among the greatest challenges that limit the effectiveness of colonoscopies.

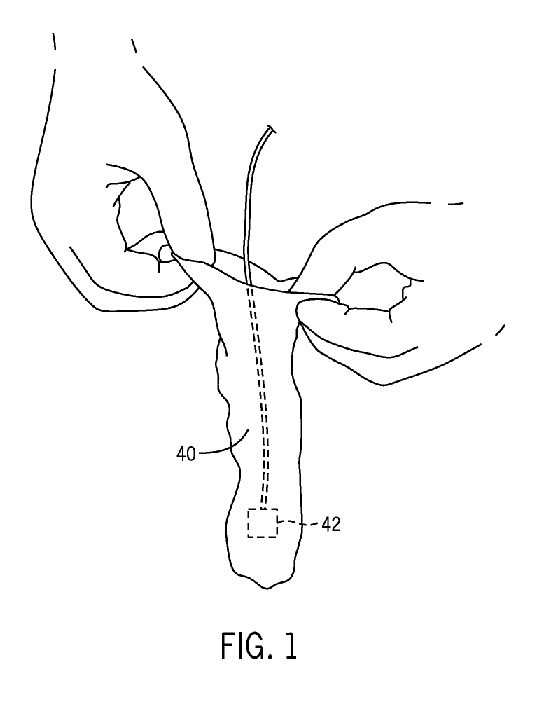

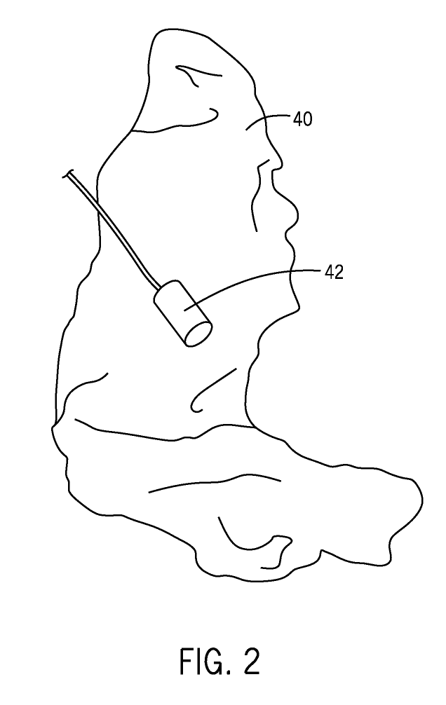

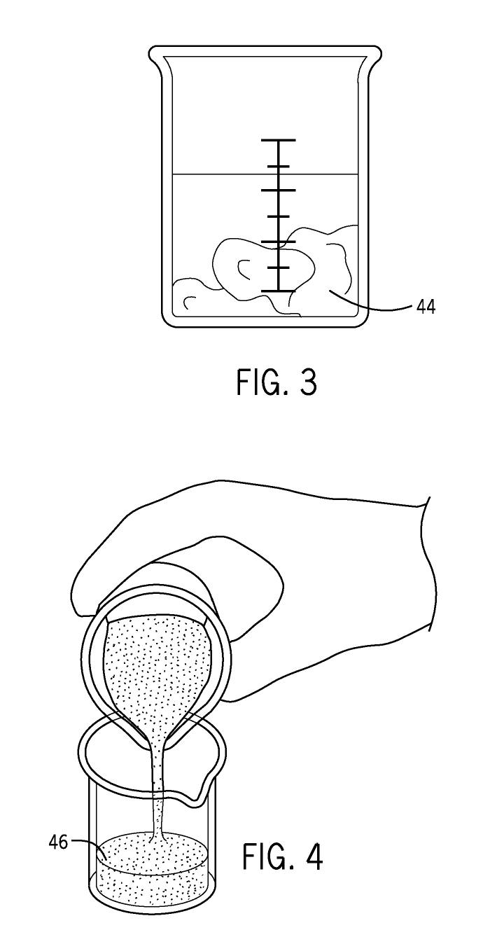

It has been shown experimentally that ultrasound can significantly increase the liquefaction of fecal matter over traditional water spray equipped devices. Others have demonstrated similar effects of the safe liquefaction on thrombus formations in the arteries and veins of pigs and humans. FIGS. 1 and 2 experimentally illustrates a typical ultrasound-liquefaction experiment in colorectal tissue 40. A transducer 42 placed inside of the porcine colon delivers ultrasound circumferentially around the placement site. FIGS. 3 and 4 shows the liquefaction of stool 44 that is obtained without ultrasound (FIG. 3) and with ultrasound (FIG. 4). Broadband ultrasound may be used, and depending on parameters such as pulse rate, acoustic intensity, duration, frequency, pulse length, and duty cycle, an increase in liquefaction speed by a factor of 50 and 100 times can be obtained. In fact, the effect of ultrasound can be so profound that solid stool samples 44 in saline can turn into a fully dispersed liquid sludge 46 within seconds (see FIG. 4).

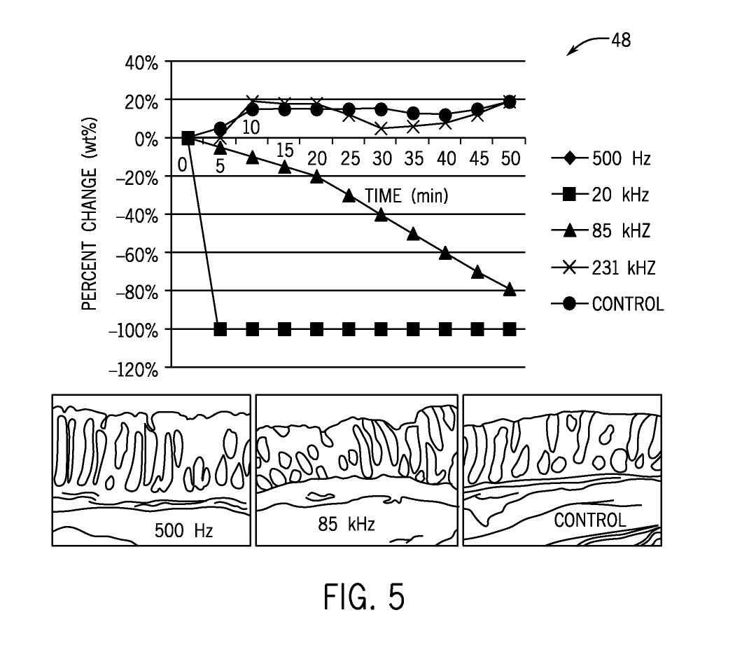

Results clearly show that ultrasound exposure assists the liquefaction of fecal matter. Furthermore, increased acoustic intensity and lower frequency correspond to enhanced dissolution of stool. FIG. 5 shows temporal percent weight change plots 48 for 1.5 g porcine stool samples and colorectal sections without ultrasound exposure (water spray control), and those exposed to 50-500 kPa of ultrasound. Control and 231 kHz samples swell and increase in weight. Sub 85 kHz and 50 kPa exposure to ultrasound increases liquefaction by greater than 50 times.

The inventors have found that ultrasound can clean the colon wall and can also be used to detect and evaluate target lesions. Controlling the shape of ultrasound focal areas inside the colon by conventional methods of ultrasound focusing, such as multi-element phased arrays, is an extremely difficult and challenging task. The complex shape of the colon as well as motion of the colonoscope makes accurate focusing of ultrasound to a target area practically impossible. A solution to this problem has been found and can be based on the use of TRA principles to control ultrasound focusing in the colon so that only regions of interest are exposed to predetermined levels of ultrasound. TRA can focus ultrasound accurately within complex shapes using simpler means than conventional phased array focusing systems, although phased array systems can be used.

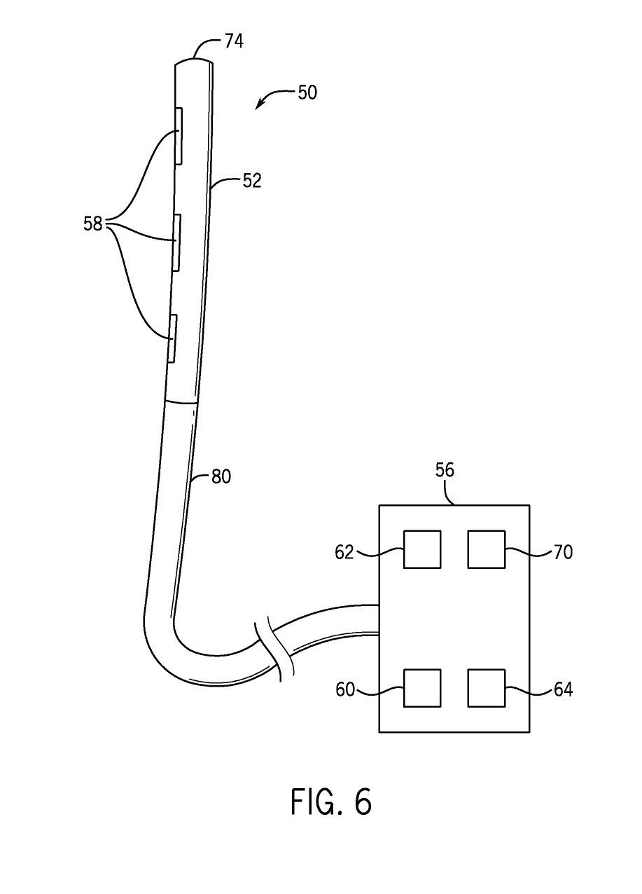

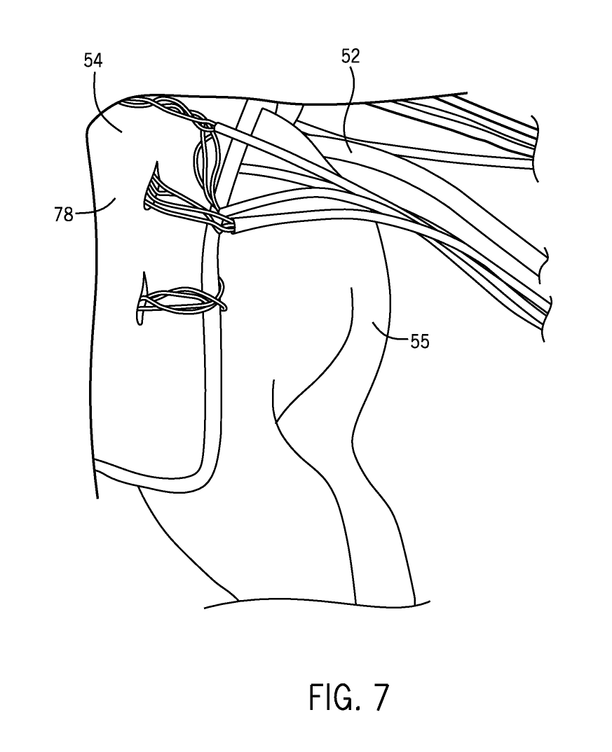

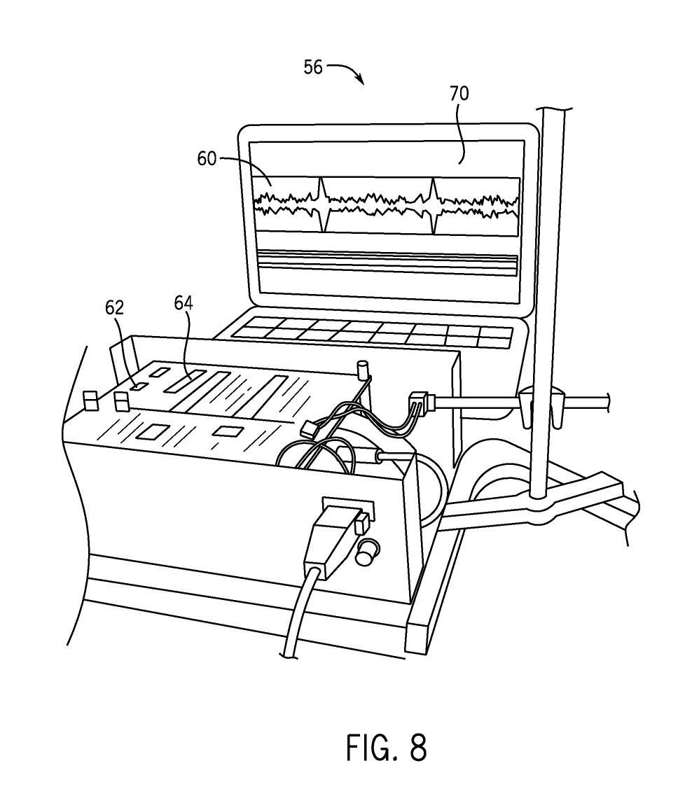

To utilize the benefits of TRA, and referring to FIGS. 6, 7, and 8, embodiments described herein provides a colonoscopy system 50. The colonoscopy system 50 can include a colonoscope 52, one or more external reverberator(s) 54 (see FIG. 7), and a controller 56 (see FIGS. 6 and 8). Each of these components will be described in greater detail below.

As described above, extraporeal ultrasound focused with TRA to the colonoscope 52 may be used diagnostically. The colorectal tissue typically has consistent echogenicity and standard B-mode, and color flow Doppler ultrasonography can provide detailed examination of colonic walls, duodenal walls, and duodenal folds. Deeply located abdominal structures such as the liver, gall bladder, spleen, pancreas, duodenum, colon, and kidneys may also be evaluated with an ultrasound equipped colonoscope. In cases where abnormalities arise in the tissue, the ultrasound from the transducer and its reflection from the sample cause spectral shifts of the ultrasound wave. As a result, there are adjacent regions of high- and low-echogenicity whose spacing depends on the size of the lesion. Using the colonoscope 52, spectral shifts can be detected readily with relatively simple means applying time of flight, Hilbert transform echo packing and Fourier domain analysis on the shear-wave created by a TRA focus. This elastography approach can allow the detection of abnormalities in the colon wall, and provide mechanical tissue properties. Thus, the systems and methods described herein are well adapted to use ultrasound focusing, to create controlled ultrasound fields across the entire colonoscope to assist the endoscopist in polyp detection, particularly those that are difficult to detect visually (flat polyps or behind folds).

The colonoscopy system 50 can also be used to measure colon wall elastography properties by monitoring the shear wave progression from a TRA “tissue-push” to tissue (i.e., an acoustic pressure strong enough to deform tissue and to generate shear waves) along the colonoscope length real-time.

Embodiments of the invention provide a new way to accurately focus ultrasound delivered through the abdominal wall to create focal volumes tailored to the shape of the colonoscope 52 and/or other targets in the colorectal region. The systems and methods proposed can exploit colonoscopy strategies that use water spray with an intraluminal port, which is typical in practice, especially for the standard colorectal examination. Embodiments of the invention can also employ the colonoscope strategy using an ultrasound stool liquefaction technique described in “Colonoscopy Systems and Methods,” WO 2012/031130, which is herby incorporated by reference.

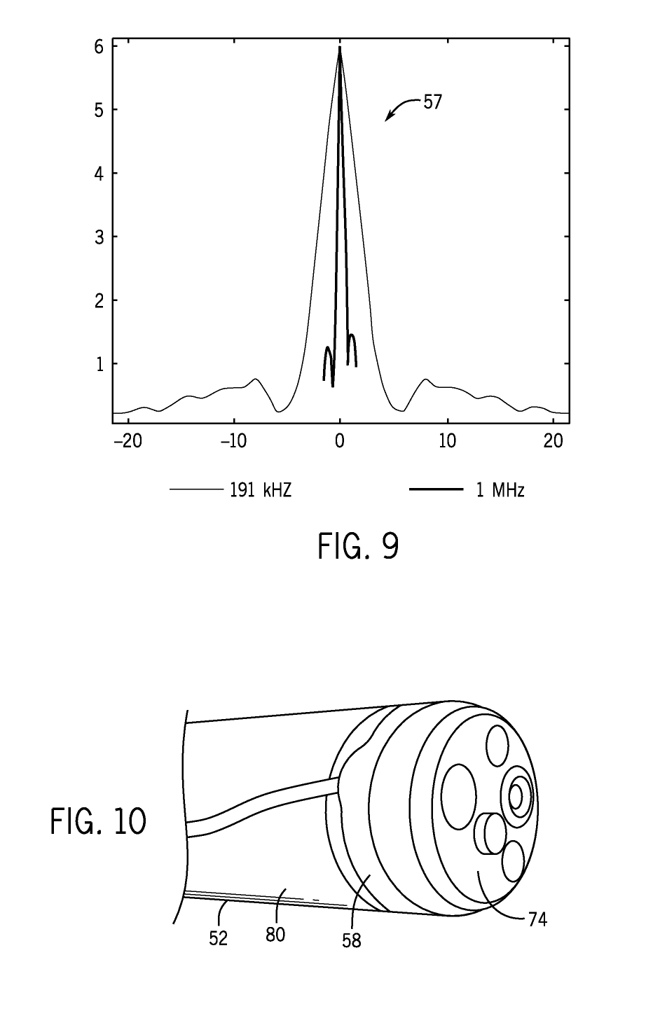

TRA focusing is based on the reversibility of acoustic propagation, which implies that the time-reversed version of an incident pressure field naturally refocuses on its source. Remarkably, numerous reflections from boundaries and internal structures, which can greatly limit and even completely diminish conventional focusing, can lead to improvement of focusing in a TRA system. The colonoscopy system 50 with a small group of piezotransducers can provide focusing with accuracy close to the diffraction limit, which would be challenging to achieve conventionally even with a costly phased array containing hundreds of elements. FIG. 9 shows TRA focusing in the colon of a female 48 kg porcine cadaver model 55 producing a focal spot 57 that is close to symmetrical. The challenge then becomes coupling this innovative focusing capability with targeted therapy and diagnostics.

TRA focusing relies on obtaining an initial signal from the target area. This requires a beacon, such as a hydrophone, located within the target region. The colonoscope 52 can serve to provide the one or more beacons 58 (see FIGS. 6 and 10). The beacons 58 can comprise one or more thin polyvinylidene fluoride (PVDF) transducer(s) 58 placed around its shaft 80 to serve as TRA beacons and provide the necessary feedback signals to establish the TRA relationship between the external reverberator 54 and the colonoscope 52. In some embodiments, the transducers 58 can be built into the colonoscope 52, or they can be built into a disposable sheath that can be placed on a traditional colonoscope. Thus, a passive colonoscope with only water spray can be enhanced by an active colonoscope 52 with TRA focusing capability.

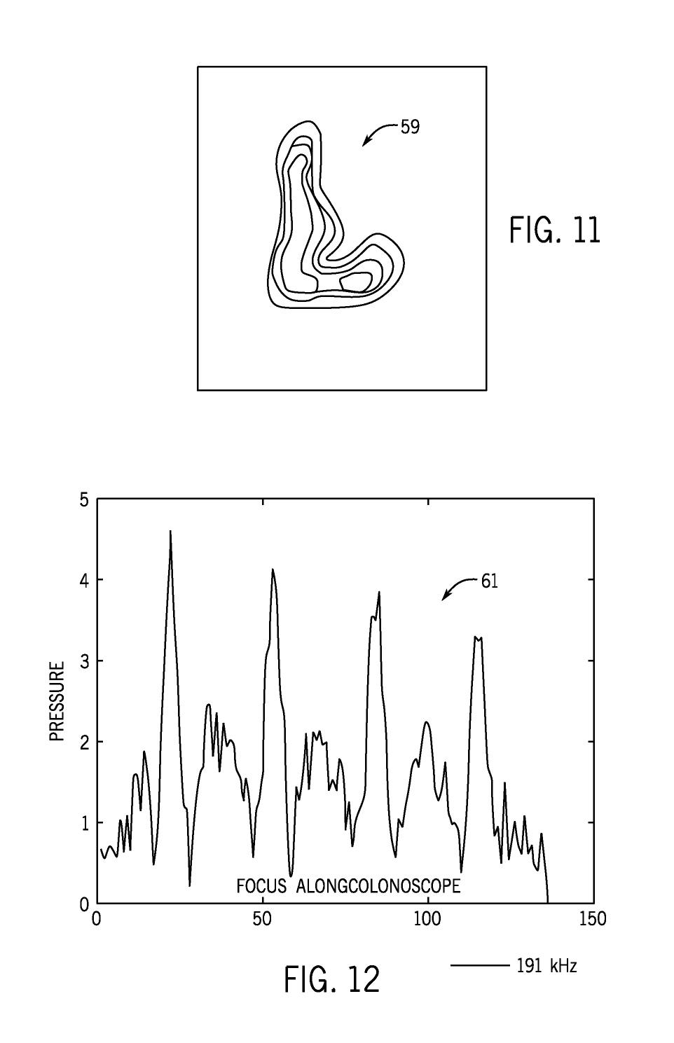

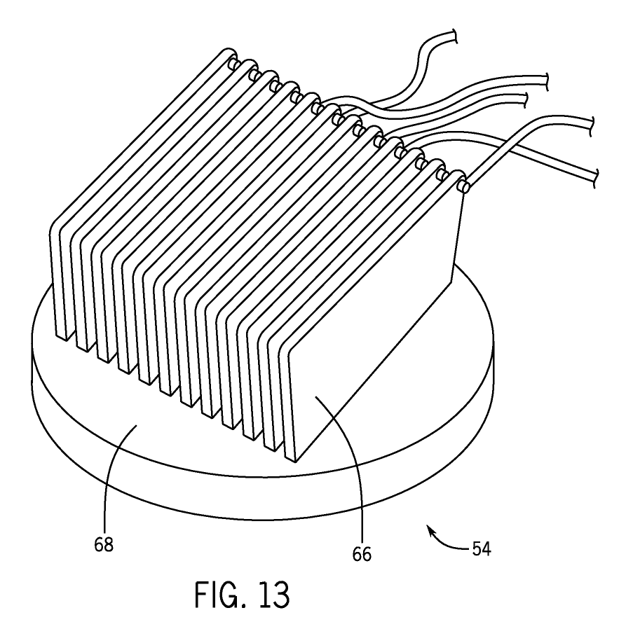

These developments set the stage for the major innovations in the use of a colonoscope—to use TRA focusing to create focal volumes of arbitrary shape that can match the colorectal physiology and to show that ultrasound can prepare the bowel and diagnose problematic quadrants along the colonoscope insertion path. The creation of focal spots of desired shape is possible by superimposing time-reversed impulse responses at several points within the volume where ultrasound can be focused. This can be achieved along the entire length of the colonoscope 52 by configuring it with multiple PVDF transducers 58, for example (see FIG. 6). The PVDF transducers can be 20 μm transducers, although other sizes are considered. Recording a set of impulse responses along the path of the colonoscope can provide a library of signals, enabling creation of the focal spot of desired 3D shape. The feasibility of forming composite focal patters 59 with complex shapes was demonstrated in previous studies (see FIG. 11) and along a colonoscope in the swine colon 61 (see FIG. 12). The colonoscope 52 can be used to measure the TRA impulse response at several different points chosen to reflect the desired focal region within the colon. Once these shapes are formed in media, e.g., stool, water, tissue etc. surrounding the colonoscope 52, and delivered during a colonoscopy procedure, they can rapidly liquefy fecal matter within the acoustic focal volumes. TRA spectral processing can then be used across the focal volume to measure the mechanical properties of the surrounding colon tissue.

Another aspect of the colonoscopy system 50 is the controller 56. The controller 56 can comprise a computer-controlled analog and/or digital controller input/output system, for example. The controller 56 and LabView software 60, for example, can be used to control and monitor a power amplifier 62, reverberator(s) 54, and ultrasound-focusing beacon(s) 58. The number of transmitting channels in the colonoscopy system 50 can be varied from 1 to 8, for example, or more, where increasing the number of transmitting channels from the transcutaneous reverberator can improve the focusing ability of the TRA system and can provide flexibility in controlling the size and the shape of the focal volume.

A waveform generator 64 of the colonoscopy system 50 can generate an initial 5-cycle excitation signal and can apply it sequentially to each channel of the reverberator 54. The controller 56 can record the signals measured from the beacon 58, which can be located at the desired ultrasound focus. The recorded signals can then be cross-correlated, time-reversed, digitized, synchronized, amplified, and applied simultaneously to one or more of the transducers of the transmitter. The resulting acoustical signals accurately focus at the target point. Software 60 can control the ultrasound exposure parameters in the treatment area such as pulse duration, pulse repetition, ultrasound on/off time, and the duration of the experiment.

The capability of TRA to focus ultrasound was measured in the colon with a miniature piezotransducer 58 attached to a colonoscope (see FIG. 10).The ultrasound therapy reverberator 54 was positioned on the lower-hind of the swine 55 and coupled to the skin with ultrasound gel (see FIG. 7). FIG. 9 shows the intensity of the 1-MHz and 191-kHz focused ultrasound as a function of position in the cadaver. In an experiment, the colonoscopy system 50 (see FIG. 6) provided 1.5 mm and 10.5 mm spatial focusing resolution, respectively in the porcine colon with a signal to noise ratio (SNR) of approximately 7, and similar results were found in the rodent skull and porcine cadaver vein as well. A multi-point complex focus 61 along the colonoscope in the porcine colon is shown in FIG. 12 at 191 kHz.

The reverberators 54 can be made of rexolite due to low ultrasound attenuation factors and accurate machinability. Other known materials may also be used. Peizoceramic (PZT-4) transducers 66 can be coupled, e.g., glued, in parallel with light-cured cyanoacrylic matching layers (EBL Products) to the facets of the rexolite lens 68 as shown in FIG. 13. Frequencies of the TRA transmitters can cover the appropriate range for therapeutic and/or diagnostic ultrasound. Due to the length of the colonoscope 52 and the multiple focusing zones, a 64 channel TRA focusing unit with 8-16 independent beacons, for example, can be used.

A user interface 70 can be used for acquiring multiple spatial TRA impulse measurements from the multiple channels and beacons 58 of the colonoscope 52. The software 60 can allow for selecting the appropriate impulse points from a library of stored signals, superimposing the stored signals, time-reversing them, and retransmitting the acoustic signature to create the desired complex focus.

The colonoscopy system 50 exposimetry calibration and focusing capability can be obtained using a beam scanner (Velmex). Calibrated needle hydrophones (Precision Acoustics and Onda) can be used to determine the ultrasonic sensitivity of the colonoscope 52 beacons 58 at the various transmitter frequencies. The calibrated colonoscope 52 can be capable of providing real-time acoustic pressure feedback from the colorectal lumen. The broad-band PVDF transducers 58 can provide sensitivity to detect cavitation using the frequency spectrum measurement approach that can be employed in the user interface 70 of the colonoscopy system 50.

Methods according to embodiments of the invention provide that ultrasound fields of desired shape can be generated inside a lumen, e.g. a colon, using an external ultrasound transmitter (reverberator 54) operating in the TRA mode. Using the controller 56, and the specially designed colonoscope 52 with multiple transducers 58, e.g., miniature paper-thin, mounted along its length that act as beacons to focus ultrasound, ultrasound fields of complex shape can be measured along the length of the colonoscope. To generate the information required for TRA focusing of the ultrasound, ultrasound impulse responses can be measured along the path of the colonoscope 52 in the colon 72 of a subject. Multiple impulse responses per beacon can be acquired as needed depending on the complexity of the desired focusing pattern. Software 60 can be used with the system 50 to enable multiple-beacon focusing and real-time acoustic shear-wave elastography processing.

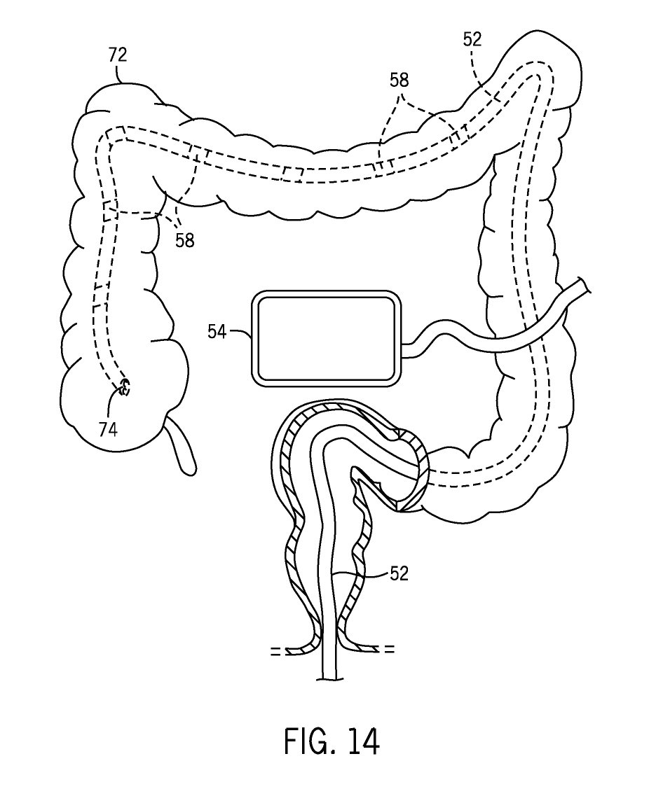

The colonoscope 52 can be attached to a water spray/imaging system (not shown) (Olympus OFP2/GIF-160, for example) and connected to the controller 56. Before insertion through fecal matter and in preparation for initial liquefaction, the colonoscope 52 can be initialized for a burrow focusing routine. Here, ultrasound can be focused only to the tip 74 of the colonoscope 52 for the rapid dissolution of densely packed solid stools as it is moved forward though the colon. To show how the magnitude of imposed ultrasonic pressure and water jet affects liquefaction of the fecal matter, the volumetric flow rate through the colonoscope 52 can be varied from 100 to 300 mL/min. At the end of the insertion, the entire length of the colonoscope 52 can be focused for through colorectal cleaning of remaining fecal debris as shown in FIG. 14.

Upon full colonoscope 52 insertion, TRA ultrasonic foci can be targeted to each beacon 58 creating a tissue push in the region of the beacon. The resultant shear wave can then be measured on nearby beacons along the length of the colonoscope. By monitoring the shear wave progression, stress/strain and regional density, the elastic modulus and viscoelasticity can be obtained. A 3D elastogram scan along the entire length of the colonoscope can be completed in relatively short time since focusing is extraporeal to the detector transducers on the colonoscope 52.

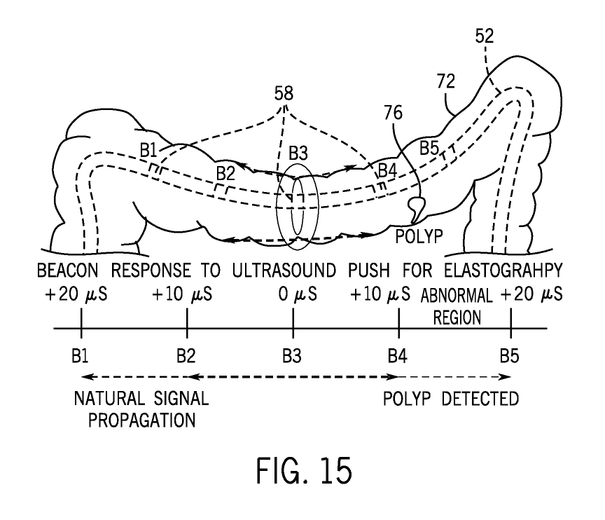

FIG. 15 shows the qualitative results that can be obtained with the elastogram and lesion formation detection. The elastogram can first be distorted on the right side of the colonoscope 52 with an emulated polyp 76 in the colorectal tissue. The elastogram can then be distorted at multiple foci to emulate multiple lesions. In an experiment, non-cadaver hydrogel-based phantom models of a colon with polyps can be constructed from gelatin, agarose and carrageenan of various concentrations (5-40 percent) to evaluate the colonoscope 52 elastogram sensitivity and specificity.

Mechanisms for ultrasound action include direct acoustic streaming, bubble generation that enhances streaming, generalized peristaltic mechanisms of TRA pulses, and effects of large shear wave attenuations and wavelengths that are comparable in size to heterogeneities in the fecal debris. Peak-pressures and Fourier spectrum content of the colonoscope 52 can be monitored to provide real-time mechanical index (MI) and thermal index (TI) feedback based on models, as well as cavitation detection across all beacons 58 on the entire length of the colonoscope 52.

In some embodiments, neoprene wraps 78 can be included to go around the waist (for example) of the subject to hold the external reverberator(s) 54 in place (see FIG. 7 for example).

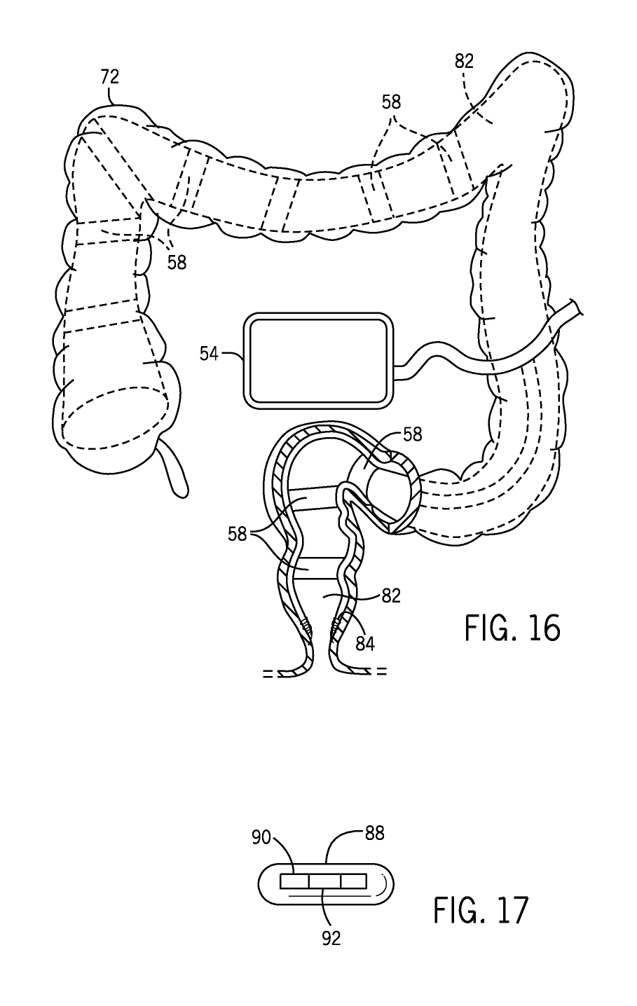

In some embodiments, a temporary sleeve 82 may be positioned in a lumen, e.g., colon 72, and remain in the lumen for a predetermined time, such as an hours, days, weeks, or months (see FIG. 16). In order to avoid migration of the sleeve within the lumen, the sleeve may be sutured in place 84. The sleeve material can be flexible, biologically inert, and permeable. Similar to the shaft 80 of the colonoscope 52, the sleeve may include a plurality of beacons 58. With an external reverberator 54 and wireless communications, the sleeve may be used in the same way as the colonoscope 52. One exemplary use may be for enhanced drug delivery by temporarily increasing intestinal permeability to molecules, or changing the chemical milieu and microbial diversity of the intestinal lumen.

In another embodiment, neoprene wraps 78 can contain multiple reverberators and can be used for extracorporeal, non-invasive lipolysis to reduce subcutaneous fat. This method, when wrapped around the torso, leg, or arms for several hours a day, allows the use of a small, portable ultrasound source to create cavitation leading to fat cell lysis. Treatment sessions can be done at home without the need to travel to an outpatient facility where bulky ultrasound equipments are currently housed. The in-home method can be wirelessly controlled and monitored by a smartphone application.

In another embodiment, a catheter configures the same as or similar to the colonoscope 52 with several ultrasound beacons 58 can be inserted into the accessory channel of a known endoscope. TRA can focus ultrasonic energy to a specific area where the catheter is directed, such as food bolus impacted in the esophagus, blood clots and organic debris in the upper and lower gastrointestinal tract, and pancreatic fluid collections. In cases of acute gastrointestinal bleeding, copious amounts of blood clots impair visualization of the source of bleeding (such as a bleeding peptic ulcer) during endoscopic visualization despite conventional water irrigation and suction. A catheter inserted through the accessory channel of an endoscope can be directed into the blood clots to dissolve and liquefy the clots, to facilitate suctioning out from the suction channel of an endoscope. Therapeutic intravascular ultrasound has been used for thrombolysis, however TRA ultrasound takes advantage of the numerous reflections from gas and fluid that is naturally inherent in the gastrointestinal tract, which may greatly limit and even completely diminish conventional focusing using conventional ultrasound, and in this environment actually enhances focusing of the TRA system. A biosensor can be used to detect and measure the intestinal wall impedance to provide a feedback mechanism so as to prevent injury from ultrasound cavitation.

In another embodiment, a wireless ultrasound capsule 88 with its own power source 90 and transducer 92 can be swallowed by a patient prior to endoscopy and can generate low frequency ultrasound in the gastrointestinal tract that can be filled with blood, fecal material, and/or other organic debris (see FIG. 17). After a period of time when liquefaction of the debris has occurred, endoscopy can now proceed safely and efficiently without the debris impairing the examination.

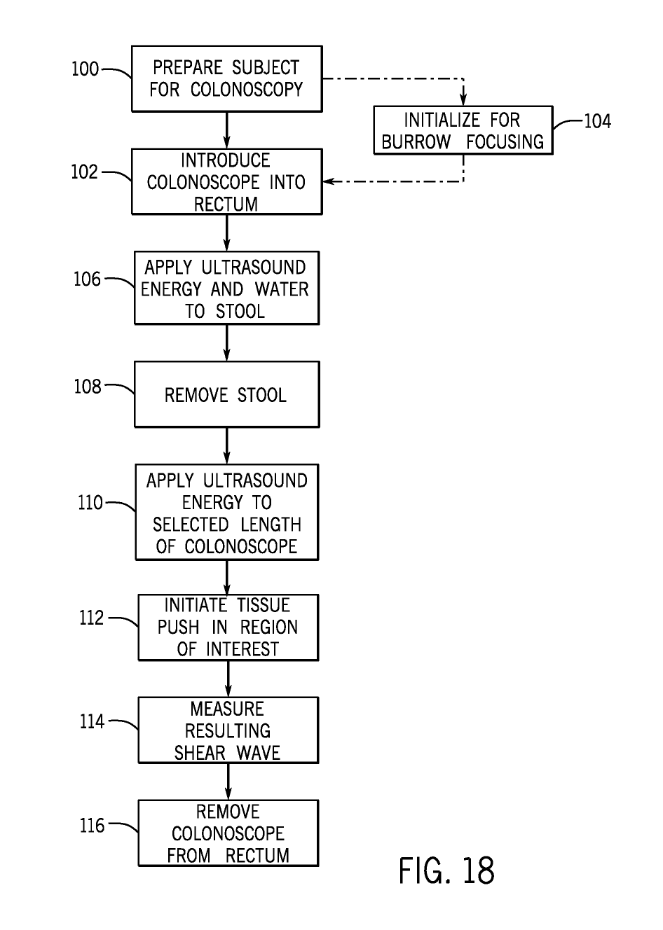

A method according to embodiments of the invention is set forth in FIG. 18. As indicated at process block 100, a subject may be premedicated and prepared for a colonoscopy. An embodiment of colonoscope 52 can be introduced into the rectum and advanced proximally, as indicated at process block 102. Optionally, at process block 104, the colonoscope 52 can be initialized for a burrow focusing routine. Here, ultrasound can be focused only to the tip 74 of the colonoscope 52 for the rapid dissolution of densely packed solid stools as it is moved forward though the colon. When stool is encountered, ultrasound energy can be applied via the ultrasound transducer without touching the stools, and may include water irrigation to aid in the stool liquefaction process, as indicated at process block 106. Stool may be removed with applied suction using conventional methods, as indicated at process block 108.

At the end of the insertion, a selected length, or the entire length of the colonoscope 52 can be focused for thorough colorectal cleaning of remaining fecal debris, as indicated at process block 110, such as until the entire mucosa of the colon segment can be seen well, and with minimal or no residual staining, small fragments of stool, or opaque liquid using a validated bowel prep scoring system.

Upon full colonoscope 52 insertion, TRA ultrasonic foci can be targeted to each beacon creating a tissue push in the region of the beacon, as indicated at process block 112. The resultant shear wave can then be measure on nearby beacons along the length of the colonoscope, as indicated at process block 114. By monitoring the shear wave progression, stress/strain and regional density, the elastic modulus and viscoelasticity can be obtained. The colonoscopy may be performed by a single operator, and may be digitally recorded. When complete, the colonoscope 52 is removed and the subject is allowed to recover, as indicated at process block 116.

The foregoing has been a detailed description of illustrative embodiments of the invention. Various modifications and additions can be made without departing from the spirit and scope thereof. Furthermore, since numerous modifications and changes will readily occur to those skilled in the art, it is not desired to limit the invention to the exact construction and operation shown and described. For example, any of the various features described herein can be combined with some or all of the other features described herein according to alternate embodiments. While the preferred embodiment has been described, the details may be changed without departing from the invention, which is defined by the claims.

Finally, it is expressly contemplated that any of the processes or steps described herein may be combined, eliminated, or reordered. In other embodiments, instructions may reside in computer readable medium wherein those instructions are executed by a processor to perform one or more of processes or steps described herein. As such, it is expressly contemplated that any of the processes or steps described herein can be implemented as hardware, software, including program instructions executing on a computer, or a combination of hardware and software. Accordingly, this description is meant to be taken only by way of example, and not to otherwise limit the scope of this invention.

We claim:

1. A medical device comprising:
a flexible tube having an operable portion insertable into a body cavity and a control end;
at least one ultrasound transducer positioned on or in the flexible tube at or near the operable portion of the flexible tube, the ultrasound transducer to generate ultrasound energy;
at least one external reverberator operable in a time reversal acoustic mode; and
an ultrasound generator circuit to control the at least one ultrasound transducer and the at least one external reverberator, the ultrasound generator circuit coupled to the at least one ultrasound transducer and the at least one external reverberator.

2. The medical device according to claim 1,
wherein the at least one ultrasound transducer comprises a polyvinylidene fluoride transducer.

3. The medical device according to claim 1,
wherein the at least one ultrasound transducer is built into the flexible tube.

4. The medical device according to claim 1,
wherein the at least one ultrasound transducer is built into a disposable sheath positionable over the operable portion of the flexible tube.

5. The medical device according to claim 1,
wherein the at least one ultrasound transducer is operable to focus ultrasound from the at least one external reverberator.

6. The medical device according to claim 1,
wherein the at least one external reverberator includes a plurality of transmitting channels.

7. The medical device according to claim 6,
further including a waveform generator, the waveform generator operable to generate an excitation signal, and apply the excitation signal sequentially to each of the plurality of transmitting channels.

8. The medical device according to claim 1,
wherein the medical device is operable for at least one of drug delivery by increasing permeability of tissue, thermal ablation and/or emulsification of tissue, location of tumors, ulcers, and other abnormalities, and diagnostic imaging.

9. A colonoscopy system comprising:
a colonoscope having a length, the colonoscope including an ultrasound beacon along the length;
a controller operatively coupled to the colonoscope, the controller including control software and a power amplifier, the control software operable to control ultrasound exposure parameters in a colon treatment area;
a reverberator operatively coupled to the controller, the reverberator operable in a time reversal acoustic mode; and
the ultrasound beacon operable to focus ultrasound from the reverberator.

10. The colonoscopy system according to claim 9,
further including an operator interface.

11. The colonoscopy system according to claim 9,
wherein the reverberator comprises a lens and peizoceramic transducers.

12. The colonoscopy system according to claim 9,
wherein the reverberator is an extracorporeal reverberator.

13. The colonoscopy system according to claim 9,
wherein the reverberator is positioned within a wrap that is positioned around a subject.

14. The colonoscopy system according to claim 9,
wherein the control software is operable to enable multiple-beacon focusing.

15. The colonoscopy system according to claim 9,
wherein the control software is operable to process real-time acoustic shear-wave elastography.

16. A method for performing a diagnostic and therapeutic endoscopy or colonoscopy, the method comprising:
introducing a flexible tube having an operable end insertable into a gastrointestinal tract, the flexible tubing including an ultrasound transducer positioned at or near the operable end of the flexible tube, and at least one water flow channel to deliver water to the gastrointestinal tract, the ultrasound transducer to generate ultrasound energy;
applying ultrasound energy to debris located in the gastrointestinal tract;
liquefying the debris via the ultrasound energy and water;
removing the liquefied debris from the gastrointestinal tract;
using time reversal acoustics, focusing ultrasound energy to the ultrasound transducer; and
measuring mechanical properties of tissue in the gastrointestinal tract for a diagnostic purpose.

17. The method according to claim 16,
further including delivering water through the at least one water flow channel to the gastrointestinal tract; and
liquefying the debris via the ultrasound energy and water.

18. The method according to claim 16,
further including initiating a tissue push in a tissue region of interest.

19. The method according to claim 18,
further including measuring a resultant shear wave on an additional ultrasound transducer positioned on the flexible tube.

20. The method according to claim 16,
performing the colonoscopy or endoscopy without the use of a colon prep or for partial colon prep.

21. The method according to claim 16,
further including evaluating abdominal structure including at least one of a liver, a gall bladder, a spleen, a pancreas, a duodenum, and a kidney.
